(19) United States
(12) Patent Application Publication
(10) Pub. No.: US 2003/0023210 A1
(43) Pub. Date: Jan. 30, 2003

(54) Disposable insert for ostomy pouch

(76) Inventors: Beatrice W. Bedard, Dayton, OH (US); Shubhangi D. Ballal, Dayton, OH (US)

Correspondence Address: Killworth, Gottman, Hagan & Schaeff, L.L.P., Suite 500, One Dayton Centre, Dayton, OH, 45402-2023, US

(21) Appl. No.: 10/208,544

(22) Filed: Jul. 30, 2002

Related U.S. Application Data
(60) Provisional application No. 60/308,642, filed on Jul. 30, 2001.

(51) Int. Cl. A61F 5/44

(52) U.S. Cl. 604/332

(57) ABSTRACT

An apparatus and system for collecting intestinal effluent. The apparatus comprises an ostomy pouch and an insert defining an interior volume. The insert is configured for positioning within the ostomy pouch and can define a pair of handles, a pair of pleated side panels, or a pair of handles and a pair of pleated side panels. The system comprises a skin adhesive barrier wafer positioned about a stoma, an ostomy pouch in communication with the stoma, and an insert. The insert defines an interior volume and is positioned within an inner chamber of the pouch. The insert can define a pair of handles, a pair of pleated side panels, or a pair of handles and a pair of pleated side panels. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims. 37 C.F.R. §1.72(b).

### CROSS-REFERENCE TO RELATED APPLICATION

This application claims the benefit of U.S. Provisional Patent Application Serial No. 60/308,642, DISPOSABLE PLASTIC INSERT FOR OSTOMY POUCH, filed Jul. 30, 2001.

### BACKGROUND OF THE INVENTION

The present invention relates generally to ostomy pouching systems and, more particularly, to a disposable insert for an ostomy pouch that is economical, sanitary, and convenient to use.

An ostomy is a surgical procedure that can be performed on the human gastrointestinal and/or urinary tract. Generally in an ostomy, the surgeon creates a permanent opening or “stoma” in the abdominal wall. A terminal end of the gastrointestinal or urinary tract is then brought to the external surface of the skin. This artificial opening allows for the discharge of bodily waste or other effluent to the outside of the body, bypassing any damaged tissue or removed section of the tract. Ostomies can be performed to the urinary tract (termed an “urostomy”), to the colon (termed a “colostomy”), or to the ileum, a section of the small intestine (termed an “ileostomy”).

After undergoing an ostomy procedure, waste from the body is no longer discharged through the normal anatomical terminus, be that the urethra and/or anus. Bodily waste instead exits the body at the stoma, which was created in the abdominal wall. An ostomy pouch is therefore employed to collect the effluent. One such ostomy pouch is the two-piece pouching system available from ConvaTec (Princeton, N.J.), which includes a plastic pouch and a skin adhesive wafer that is coupled with the pouch.

The above-described pouch is designed to be disposable. However, with an ostomy, urinary and/or intestinal continence is physiologically impossible. Therefore, as bodily waste is excreted from the body and through the stoma, the pouch fills and must be repeatedly emptied. Given that the pouch must often be emptied several times daily, the economic impact of continuously replacing used pouches makes the disposable aspect of these pouches impractical. As a result, many ostomy patients must resort to reusing filled pouches. The pouch must therefore be emptied and rinsed clean prior to reapplication. This process is unsanitary, odoriferous, inconvenient, and quite time consuming, given the diminished dexterity of many ostomy patients.

Accordingly, the present inventors have recognized a need for improvements in ostomy pouching system design.

### SUMMARY OF THE INVENTION

The present invention meets this need by providing an apparatus for collecting intestinal effluent. Although the present invention is not limited to specific advantages or functionality, it is noted that the apparatus provides a convenient, sanitary, and cost effective alternative to the use of ostomy pouches in the traditional manner. The present invention enables the ostomy patient to reuse the relatively expensive ostomy pouch without having to cleanse the pouch after discarding its contents. Consequently, the apparatus provides an enhanced ostomy pouching system which is economical, less odoriferous, and can be changed relatively easily.

In one embodiment of the present invention, an apparatus for collecting intestinal effluent is provided comprising an ostomy pouch and an insert defining an interior volume and a pair of handles. The insert is configured for positioning within the ostomy pouch.

In another embodiment of the present invention, an apparatus for collecting intestinal effluent is provided comprising an ostomy pouch and an insert defining an interior volume and a pair of pleated side panels. The insert is configured for positioning within the ostomy pouch.

In still another embodiment of the present invention, an apparatus for collecting intestinal effluent is provided comprising an ostomy pouch and an insert defining an interior volume, a pair of handles, and a pair of pleated side panels. The insert is configured for positioning within the ostomy pouch.

In yet another embodiment of the present invention, a system for collecting intestinal effluent is provided comprising a skin adhesive barrier wafer, an ostomy pouch defining an inner chamber, and an insert defining an interior volume. The barrier wafer is positioned about a stoma and the ostomy pouch is in communication with the stoma. The insert is positioned within the inner chamber of the ostomy pouch and is configured to collect the intestinal effluent that flows from the stoma. The insert defines a pair of handles.

In still yet another embodiment of the present invention, a system for collecting intestinal effluent is provided comprising a skin adhesive barrier wafer, an ostomy pouch defining an inner chamber, and an insert defining an interior volume. The barrier wafer is positioned about a stoma and the ostomy pouch is in communication with the stoma. The insert is positioned within the inner chamber of the ostomy pouch and is configured to collect the intestinal effluent that flows from the stoma. The insert defines a pair of pleated side panels.

In still yet another embodiment of the present invention, a system for collecting intestinal effluent is provided comprising a skin adhesive barrier wafer, an ostomy pouch defining an inner chamber, and an insert defining an interior volume. The barrier wafer is positioned about a stoma and the ostomy pouch is in communication with the stoma. The insert is positioned within the inner chamber of the ostomy pouch and is configured to collect the intestinal effluent that flows from the stoma. The insert defines a pair of handles and a pair of pleated side panels.

These and other features and advantages of the invention will be more fully understood from the following detailed description of a typical embodiment of the present invention, taken together with the accompanying drawings, and the appended claims. It is noted that the scope of the claims is defined by the recitations therein and not by the discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 is an illustration of an insert in accordance with one embodiment of the apparatus for collecting intestinal effluent of the present invention;

FIG. 2 is an illustration of a skin adhesive barrier wafer for use with the system for collecting intestinal effluent of the present invention;

FIG. 3 is an illustration of an ostomy pouch for use with the system for collecting intestinal effluent of the present invention;

FIG. 4 is an illustration of an insert in accordance with one embodiment of the present invention in conjunction with an ostomy pouch; and

FIG. 5 is an illustration of an insert in accordance with another embodiment of the present invention defining an enlarged opening in a bottom portion of an ostomy pouch.

Skilled artisans appreciate that elements in the figures are illustrated for simplicity and clarity and have not necessarily been drawn to scale. For example, the dimensions of some of the elements in the figures may be exaggerated relative to other elements to help improve understanding of embodiment(s) of the present invention.

### DETAILED DESCRIPTION OF THE EMBODIMENTS OF THE INVENTION

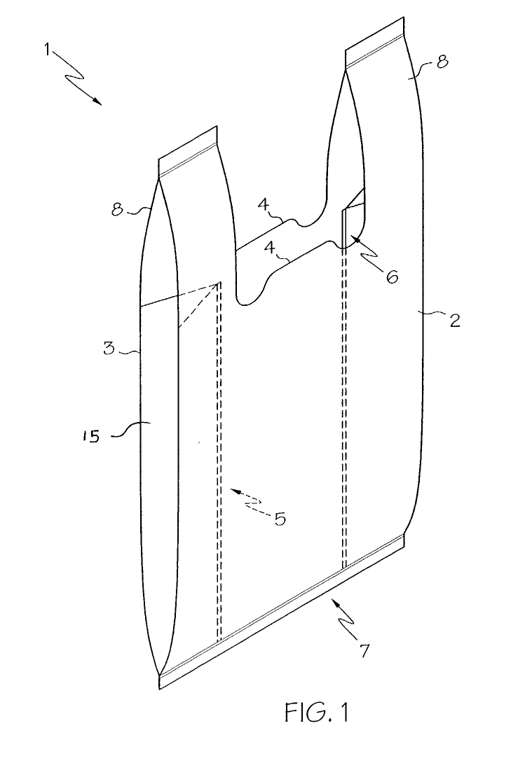

In accordance with one embodiment of the present invention, an apparatus for collecting intestinal effluent is provided. The apparatus includes an ostomy pouch and an insert 1 defining an interior volume. The insert 1, which is illustrated in FIG. 1, is configured for positioning within the ostomy pouch and can be comprised of two plastic sheets 2,3. The sheets 2,3 each define a top edge 4, a pair of side edges 5,6, and a bottom edge 7. The pair of side edges 5,6 and the bottom edge 7 can be heat sealed or extruded to form the interior volume. The top edge 4 defines an opening to the interior volume. Moreover, the insert 1 defines a pair of handles 8 at the top edge 4.

The insert 1 is designed to be disposable and can be comprised of a pliable polymeric material, such as, for example, a polyethylene material that can have a density of about 1 mil. The polymeric material can be transparent. In addition, the ostomy pouch can define a bottom portion that defines an enlarged opening. The bottom edge 7 of the insert 1 can extend through the enlarged opening.

In accordance with another embodiment of the present invention, an apparatus for collecting intestinal effluent is provided comprising an ostomy pouch and an insert 1 defining an interior volume and a pair of pleated side panels 15. The insert 1, which is also illustrated in FIG. 1, is configured for positioning within the ostomy pouch. The present embodiment of the invention can be comprised of two plastic sheets 2,3. The sheets 2,3 each define a top edge 4, a pair of side edges 5,6, and a bottom edge 7. The pair of side edges 5,6 and the bottom edge 7 can be heat sealed or extruded to form the interior volume. The top edge 4 defines an opening to the interior volume. The pleated side panels 15 expand the interior volume of the insert 1 and are defined at the side edges 5,6.

The insert 1 is designed to be disposable and also can be comprised of a pliable polymeric material, such as, for example, polyethylene material having a density of about 1 mil. The polymeric material can be transparent. This embodiment of the invention can also comprise an ostomy pouch which defines a bottom portion with an enlarged opening. The bottom edge 7 of the insert 1 can extend through the enlarged opening.

In accordance with still another embodiment of the present invention, an apparatus is provided that comprises an ostomy pouch and an insert 1 defining an interior volume, a pair of handles 8, and a pair of pleated side panels 15. The insert 1 is configured for positioning within the ostomy pouch.

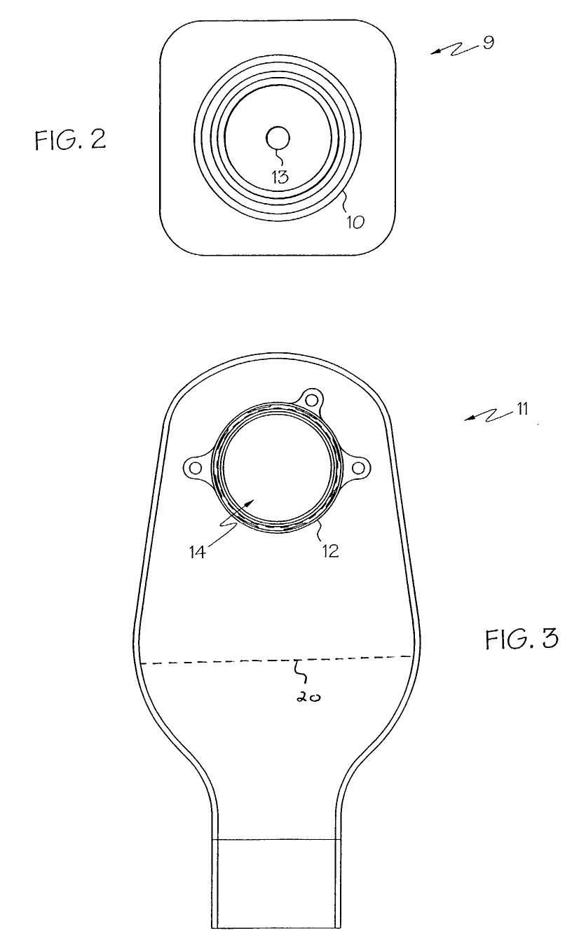

The present invention, which lines the inner chamber of an ostomy pouch, provides a superior means for collection of effluent from the body. Typically, the insert 1 is used with a two-piece ostomy pouching system, such as, for example, the SUR-FIT® Natura™ Ostomy Management System available from ConvaTec (Princeton, N.J.). In accordance with yet another embodiment of the present invention, a system for collecting intestinal effluent is provided. As is illustrated in FIGS. 2 and 3, the system comprises a skin adhesive barrier wafer 9 positioned about a stoma and an ostomy pouch 11 in communication with the stoma. The pouch 11 defines an inner chamber. The barrier wafer 9 comprises a wafer flange 10 that defines a wafer opening 13 positioned at the center of the barrier wafer 9 and wafer flange 10. A corresponding pouch flange 12, with a pouch opening 14 positioned at the center of the pouch flange 12, is positioned at a top portion of the ostomy pouch 11. The wafer opening 13 and the pouch opening 14 are configured for receipt of intestinal effluent.

Ordinarily, without the insert I of the present invention, the skin adhesive barrier wafer 9 is first adhered to the skin at the site of the stoma. The pouch flange 12 of the ostomy pouch 11 is then coupled with the wafer flange 10, which is attached to the wafer 9. As digestive functions progress, effluent from the body passes from the stoma, through the wafer opening 13 of the skin adhesive barrier wafer 9 and though the pouch opening 14, and into the ostomy pouch 11. The pouch 11 can define either a closed end or an open end at a bottom portion of the pouch 11. The open end allows the escape of collected effluent.

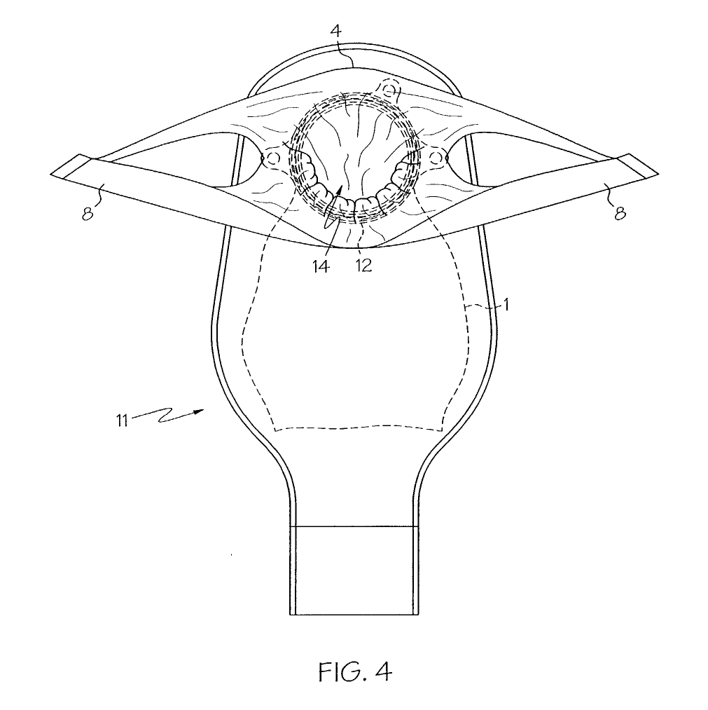

In the instant embodiment of the invention, an insert 1 is provided that defines an interior volume. The insert 1 is positioned within the inner chamber of the ostomy pouch 11 and is configured to collect the intestinal effluent that flows from the stoma. The insert 1 defines a pair of handles 8, which can be used for removing the insert 1 from the pouch 11 and carrying the insert 1 for disposal of collected effluent. As is illustrated in FIG. 4, the insert 1 is positioned such that the top edge 4 and the handles 8 extend though the pouch opening 14 of the ostomy pouch 11. The top edge 4 and the handles 8 can extend about 1 inch through the pouch opening 14. With the insert 1 positioned in such a manner, the pouch flange 12 is coupled with the wafer flange 10 of the skin adhesive barrier wafer 9. The wafer flange 10 is coupled with the pouch flange 12 by fitting inside of the pouch flange 12, thereby sealing the top edge 4 of the insert 1 between the wafer flange 10 and the pouch flange 12. Therefore, as intestinal effluent flows from the stoma and though the wafer opening 13 and the pouch opening 14, it collects within the interior volume of the insert 1, leaving the surrounding ostomy pouch 11 completely free from direct contact with bodily waste (the intestinal effluent).

In addition, the interior volume of the insert 1 is greater than or equal to the volume of the inner chamber of the ostomy pouch 11. This feature enables the ostomy patient to address sudden elimination of large amounts of unformed and/or formed intestinal effluent and, in emergency situations, to go longer periods without having to empty the contents of the insert 1. Moreover, given that the insert 1 does not comprise vents for release of intestinal gasses, the increased volume allows the insert 1 to expand without having to continuously release the gasses. The lack of vents also reduces odor as the gasses are contained within the insert 1 until expressed.

In accordance with still yet another embodiment of the present invention, a system for collecting intestinal effluent is provided wherein the insert 1 defines a pair of pleated side panels 15. The pleated side panels 15 are defined at the side edges 5,6 and expand the interior volume of the insert 1. In accordance with still yet another embodiment of the present invention, a system for collecting intestinal effluent is provided wherein the insert 1 defines both a pair of handles 8, and a pair of pleated side panels 15.

While some traditional plastic ostomy pouches comprise a drain and clip at the bottom of the pouch for release of collected effluent, the insert 1 of the present invention makes such features unnecessary. However, as intestinal effluent collects in the insert 1, it must eventually be emptied. The insert 1 is designed to be disposable. By using a new insert 1 in lieu of replacing the ostomy pouch 11 upon each disposal of effluent, a significant cost savings is realized, given the relative expense of the disposable insert 1 of the present invention versus that of the ostomy pouch 11. Moreover, the insert 1 of the present invention enables the ostomy patient to remove the collected effluent without having to cleanse the ostomy pouch 11.

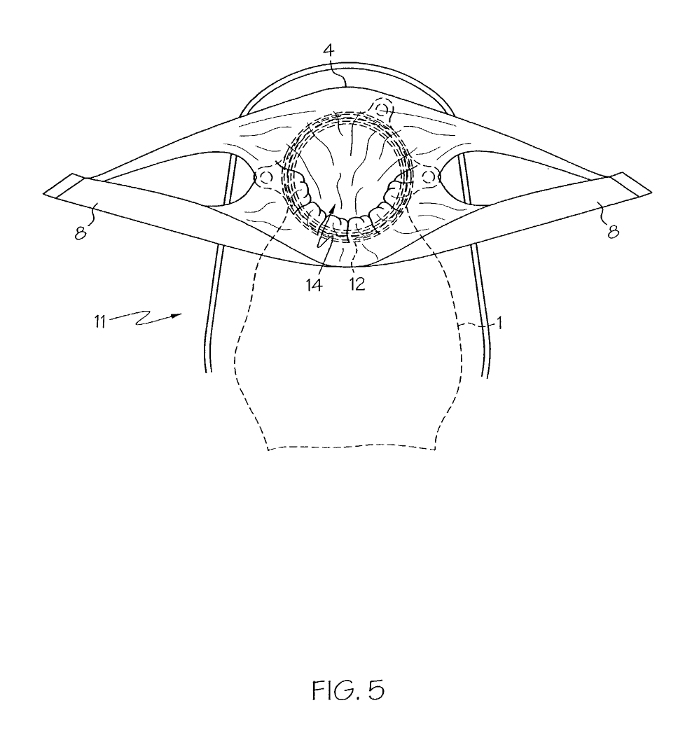

To dispose of the collected effluent, the ostomy pouch 11 is first disconnected from the skin adhesive barrier wafer 9. Next, the insert 1, which contains the collected effluent, is removed from the pouch 11 by pulling the insert 1 through the pouch opening 14 by use of the handles 8. Alternatively, the insert 1 can be removed through the bottom of the pouch 11. In order to remove the insert 1 through the bottom of the pouch 11, an enlarged opening is made by cutting a straight line 20 across the bottom portion of the pouch 11, for example, approximately 5 inches below the pouch flange 12 (see FIG. 3). This cut must be made prior to inserting the insert 1 into the pouch 11 in order to avoid piercing the insert 1. However, as is illustrated in FIG. 5, by cutting the enlarged opening in the bottom of the pouch 11, the bottom edge 7 of the insert 1 extends through the enlarged opening. This further increases the interior volume of the insert 1.

After removing the insert 1 either through the top or bottom of the pouch 11, the effluent can then be disposed of by pouring into an appropriate basin and discarding the used insert 1, or tying the handles 8 and discarding the insert 1 in a trash receptacle together with the enclosed effluent. The ostomy pouch 11 remains clean and can be immediately reused, together with a new insert 1. The insert 1 does not readily degrade in water and therefore is not designed to be flushed down a toilet.

While the invention has been described by reference to certain typical embodiments, it should be understood that numerous changes could be made within the spirit and scope of the inventive concepts described. Accordingly, it is intended that the invention not be limited to the disclosed embodiments, but that it have the full scope permitted by the language of the following claims.

We claim:

1. An apparatus for collecting intestinal effluent comprising:
an ostomy pouch; and
an insert defining an interior volume and a pair of handles, wherein said insert is configured for positioning within said ostomy pouch.

2. The apparatus of claim 1 wherein said insert comprises two plastic sheets each defining a top edge, a bottom edge, and a pair of side edges, wherein said bottom edge and said pair of side edges are heat sealed or extruded to form said interior volume, and wherein said top edge defines an opening to said interior volume.

3. The apparatus of claim 2 wherein said handles are defined at said top edge.

4. The apparatus of claim 1 wherein said insert comprises a polymeric material.

5. The apparatus of claim 4 wherein said polymeric material is pliable.

6. The apparatus of claim 4 wherein said polymeric material is transparent.

7. The apparatus of claim 4 wherein said polymeric material is polyethylene.

8. The apparatus of claim 1 wherein said insert is disposable.

9. The apparatus of claim 1 wherein said ostomy pouch defines a bottom portion, wherein said bottom portion defines an enlarged opening, and wherein a bottom edge of said insert extends through said enlarged opening.

10. An apparatus for collecting intestinal effluent comprising:
an ostomy pouch; and
an insert defining an interior volume and a pair of pleated side panels, wherein said insert is configured for positioning within said ostomy pouch.

11. The apparatus of claim 10 wherein said pleated side panels expand said interior volume of said insert.

12. The apparatus of claim 10 wherein said insert comprises two plastic sheets each defining a top edge, a bottom edge, and a pair of side edges, wherein said bottom edge and said pair of side edges are heat sealed or extruded to form said interior volume, and wherein said top edge defines an opening to said interior volume.

13. The apparatus of claim 12 wherein said pleated side panels are defined at said side edges.

14. The apparatus of claim 10 wherein said insert comprises a polymeric material.

15. The apparatus of claim 14 wherein said polymeric material is pliable.

16. The apparatus of claim 14 wherein said polymeric material is transparent.

17. The apparatus of claim 14 wherein said polymeric material is polyethylene.

18. The apparatus of claim 10 wherein said insert is disposable.

19. The apparatus of claim 10 wherein said ostomy pouch defines a bottom portion, wherein said bottom portion defines an enlarged opening, and wherein a bottom edge of said insert extends through said enlarged opening.

20. An apparatus for collecting intestinal effluent comprising:
an ostomy pouch; and
an insert defining an interior volume, a pair of handles, and a pair of pleated side panels, wherein said insert is configured for positioning within said ostomy pouch.

21. A system for collecting intestinal effluent comprising:
a skin adhesive barrier wafer positioned about a stoma;
an ostomy pouch in communication with said stoma, wherein said pouch defines an inner chamber; and
an insert, wherein said insert defines an interior volume and is positioned within said inner chamber of said ostomy pouch, wherein said insert is configured to collect said intestinal effluent that flows from said stoma, and wherein said insert defines a pair of handles.

22. The system of claim 21 wherein said barrier wafer comprises a wafer flange defining a wafer opening, and wherein said wafer opening is configured for receipt of said intestinal effluent.

23. The system of claim 22 wherein said barrier wafer and said wafer flange both define a center, and wherein said wafer opening is positioned at said center of said barrier wafer and said wafer flange.

24. The system of claim 21 wherein said ostomy pouch comprises a pouch flange defining a pouch opening, and wherein said pouch opening is configured for receipt of said intestinal effluent.

25. The system of claim 24 wherein said pouch flange defines a center, and wherein said pouch opening is positioned at said center of said pouch flange.

26. The system of claim 24 wherein said ostomy pouch defines a top portion, and wherein said pouch flange is positioned at said top portion of said pouch.

27. The system of claim 24 wherein said insert defines a top edge, wherein said handles are defined at said top edge, and wherein said top edge and said handles extend through said pouch opening.

28. The system of claims 21 wherein said barrier wafer comprises a wafer flange, wherein said ostomy pouch comprises a pouch flange, and wherein said wafer flange is coupled within said pouch flange, thereby sealing said insert between said wafer flange and said pouch flange.

29. The system of claim 21 wherein said ostomy pouch defines a bottom portion, wherein said bottom portion defines a closed end.

30. The system of claim 21 wherein said ostomy pouch defines a bottom portion, wherein said bottom portion defines an open end.

31. The system of claim 21 wherein said ostomy pouch defines a bottom portion, wherein said bottom portion defines an enlarged opening, and wherein a bottom edge of said insert extends through said enlarged opening.

32. The system of claim 21 wherein said intestinal effluent flowing from said stoma collects within said interior volume of said insert.

33. The system of claim 21 wherein said insert enables said ostomy pouch to be free from direct contact with said intestinal effluent.

34. The system of claim 21 wherein said interior volume of said insert is greater than or equal to a volume of said inner chamber of said ostomy pouch.

35. The system of claim 21 wherein said insert is disposable.

36. A system for collecting intestinal effluent comprising:
a skin adhesive barrier wafer positioned about a stoma;
an ostomy pouch in communication with said stoma, wherein said pouch defines an inner chamber; and
an insert, wherein said insert defines an interior volume and is positioned within said inner chamber of said ostomy pouch, wherein said insert is configured to collect said intestinal effluent that flows from said stoma, and wherein said insert defines a pair of pleated side panels.

37. The system of claim 36 wherein said barrier wafer comprises a wafer flange defining a wafer opening, and wherein said wafer opening is configured for receipt of said intestinal effluent.

38. The system of claim 37 wherein said barrier wafer and said wafer flange both define a center, and wherein said wafer opening is positioned at said center of said barrier wafer and said wafer flange.

39. The system of claim 36 wherein said ostomy pouch comprises a pouch flange defining a pouch opening, and wherein said pouch opening is configured for receipt of said intestinal effluent.

40. The system of claim 39 wherein said pouch flange defines a center, and wherein said pouch opening is positioned at said center of said pouch flange.

41. The system of claim 39 wherein said ostomy pouch defines a top portion, and wherein said pouch flange is positioned at said top portion of said pouch.

42. The system of claim 36 wherein said ostomy pouch defines a bottom portion, and wherein said bottom portion comprises a closed end.

43. The system of claim 36 wherein said ostomy pouch defines a bottom portion, and wherein said bottom portion comprises an open end.

44. The system of claim 36 wherein said ostomy pouch defines a bottom portion, wherein said bottom portion defines an enlarged opening, and wherein a bottom edge of said insert extends through said enlarged opening.

45. The system of claim 39 wherein said insert defines a top edge, and wherein said top edge extends through said pouch opening.

46. The system of claim 36 wherein said barrier wafer comprises a wafer flange, wherein said ostomy pouch comprises a pouch flange, and wherein said wafer flange is coupled within said pouch flange, thereby sealing said insert between said wafer flange and said pouch flange.

47. The system of claim 36 wherein said intestinal effluent flowing from said stoma collects within said interior volume of said insert.

48. The system of claim 36 wherein said insert enables said ostomy pouch to be free from direct contact with said intestinal effluent.

49. The system of claim 36 wherein said interior volume of said insert is greater than or equal to a volume of said inner chamber.

50. The system of claim 36 wherein said pleated side panels expand said interior volume of said insert.

51. The system of claim 36 wherein said insert is disposable.

52. The system of claim 36 wherein said insert defines a pair of side edges, and wherein said pleated side panels are defined at said side edges.

53. A system for collecting intestinal effluent comprising:
a skin adhesive barrier wafer positioned about a stoma;
an ostomy pouch in communication with said stoma, wherein said pouch defines an inner chamber; and
an insert, wherein said insert defines an interior volume and is positioned within said inner chamber of said ostomy pouch, wherein said insert is configured to collect said intestinal effluent that flows from said stoma, wherein said insert defines a pair of handles, and wherein said insert defines a pair of pleated side panels.
